(19) United States
(12) Patent Application Publication
(10) Pub. No.: US 2019/0144497 A1
(43) Pub. Date: May 16, 2019

(54) CONCENTRATED PROTEIN MATERIALS FROM DE-CHLOROPHYLLIZED AQUATIC PLANT BIOMASS

(71) Applicant: HINOMAN LTD., Or Yehuda (IL)

(72) Inventor: Mircea Dan BUCEVSCHI, Rehovot (IL)

(21) Appl. No.: 16/315,642

(22) PCT Filed: Jul. 6, 2017
(86) PCT No.: PCT/IL2017/050757

§ 371 Date: Jan. 6, 2019

Related U.S. Application Data
(60) Provisional application No. 62/359,242, filed on Jul. 7, 2016.

(51) Int. Cl. C07K 1/36; C07K 1/14; C07K 14/415

(52) U.S. Cl. CPC C07K 1/36; C07K 14/415; C07K 1/145

(57) ABSTRACT

A method for producing a water-soluble protein concentrate from plant biomass is disclosed, comprising: obtaining plant matter from said plant biomass; drying said plant matter; grinding said dry plant matter; de-chlorophyllizing said ground dry plant matter; treating said ground dry de-chlorophyllized plant matter with water, thereby at least partially dissolving water-soluble protein content of said dry de-chlorophyllized plant matter and preparing an aqueous suspension of said dry de-chlorophyllized plant matter; separating said aqueous suspension of said dry de-chlorophyllized plant matter into a first neutral extract and a wet solid; and, drying said first neutral extract, thereby yielding a water-soluble protein concentrate. Also disclosed is a water-soluble protein concentrate produced by the method.

### REFERENCE TO RELATED PUBLICATIONS

This application claims priority from U.S. Provisional Pat. Appl. No. 62/359,242, filed 7 Jul. 2016, and which is hereby incorporated by reference in its entirety.

### FIELD OF THE INVENTION

This invention relates in general to protein concentrates produced from dechlorophyllized plant materials and methods for producing such concentrates. It relates in particular to protein concentrates produced from dechlorophyllized leaves and fronds from aquatic plants such as duckweed and methods for their production.

### BACKGROUND OF THE INVENTION

Plants represent a renewable resource that produces biomass that can be used either directly for such uses as food or energy generation or indirectly as a source of raw materials that can be converted into any number of products such as adhesives, fibers, bioplastics, products used in the cosmetic industry, drugs, biofuels, etc. Vegetable biomass derived from plants is a composite material and comprises a wide diversity of organic and inorganic compounds. The proportions of the different chemical constituents of the biomass depend on the particular plant from which it is derived and the part of the plant that is used.

That portion of plant biomass that derives from the parts of the plant that contain chlorophyll is known as “green plant biomass.” Two general methods for processing green plant biomass are known in the art. Green juice processing uses fresh wet biomass and subjects it to mechanical operations to extract a liquid phase called “green juice” and a solid phase called “green pellet,” each of which is then processed separately. Plant extract processing produces a suspension by contact between wet or dry plant biomass and a liquid medium that can be aqueous or non-aqueous, followed by separation into a liquid phase called “plant extract” and a solid phase called “plant pellet,” each of which is then processed separately.

U.S. Pat. No. 3,173,309 discloses a method for producing a nutrient from unicellular green Chlorella algae. The algae are cultured for 72 to 96 hours. The supply of reducible carbon is then removed from the medium and the pH of the culture is adjusted until it is in the range from 8.0 to 8.5. The culture is then agitated by introduction of oxygen and decolorized by exposure to artificial white light having an intensity in excess of 5000 foot candles. After 8 to 16 hours under these conditions the chlorophyll and chlorophyll-like compounds are destroyed and the product is collected, preferably by centrifugation. The algae are then dried, preferably by lyophilization. This process yields a fluffy white or light tan powder of bland flavor and odor which may be used directly as a food supplement.

U.S. Pat. No. 4,334,024 discloses a method for preparing crystalline ribulose 1.5-bis-phosphate carboxylase from plant material that comprises grinding a sample of plant material with a suitable buffer solution; filtering the solution; adding to the solution, while stirring, sufficient quantities of polyethylene glycol (PEG) having a molecular weight in the range from 5000 to 7000 to bring the PEG to a final concentration of between 5% and 15% (w/v); discarding the precipitate; storing the solution for about 1 to 8 hours; collecting and washing the crystals formed during the storage period; and lyophilizing the crystals.

Effiong et al. (2009) discloses a method of producing a water-soluble antimicrobial agent/food preservative from duckweed (Lemna pauciscostata). Duckweed was cultured in and then harvested from outdoor concrete tanks. The harvested plant material was rinsed with clean water and evenly spread on a mosquito net-sized mesh to dry and thereafter dried in a forced air oven at 65° C. for 48 hours before being ground to a powder. The powder was exhaustively extracted with 95% ethanol and sterile distilled water at room temperature for 2 days.

Rusoff et al. (1980) discloses a method of treating duckweed. Duckweed was dried in the sun and then mixed with 12 times its weight of 0.5 N NaOH to bring the pH to above 8.5. The mixture was placed in a blender and homogenized for 60 s. The juice was then squeezed out of the homogenate through a double layer of cheesecloth and clarified by centrifugation at 2000 rpm, and the protein was precipitated from the supernatant by acidifying to pH 3.65 with 0.1 N HCl. The acidified suspension was heated to 75° C. to coagulate the soft gelatinous protein which was then refrigerated overnight. The supernatant was siphoned off and the precipitated protein was separated from the liquid portion by centrifugation at 2,000 rpm. It was subsequently frozen in thin layers in pans and dried in a Virtis freeze-dryer at −40° C. The chlorophylls, other pigments, and lipids were removed from the concentrate with boiling acetone in a Soxhlet apparatus. The concentrate was dried in a desiccator. The protein concentrate can also be obtained from washed fresh duckweed. Instead of alkalinizing with NaOH, anhydrous ammonia was bubbled through the biomass to a pH of over 8.5. The alkalinized duckweeds were then treated as described above.

Victoria González López et al (2010) and Xiangliang Pan et al. (2010) disclose additional methods for treatment of green biomass. The biomass was recovered by centrifugation (2,200 g, 5 min), washed with a 1% (w/v, g/100 mL) aqueous NaCl solution, centrifuged again and freeze-dried. The dry biomass was analyzed immediately or stored at 22° C. for up to 10 days prior to analysis. The following pretreatment methods were tested: (i) suspension in lysis buffer; (ii) ultrasonication at high power for 10 minutes using a commercial sonic bath in lysis buffer; (iii) milling for 5 minutes with a pestle and mortar without grinding elements prior to suspension in lysis buffer; and (iv) milling for 5-min with a pestle and mortar in presence of an inert ceramic powder, the grinding particles prior to suspending in the lysis buffer.

Pietryczuk A. et al. (2009) discloses a different method of soluble protein extraction from duckweed. Fresh W. arrhiza (0.1 g) was filtered and homogenized, water-soluble proteins extracted by exposing the homogenized plant material overnight to 0.1 M NaOH at 4° C., and the amount of water-soluble protein extracted then determined.

Al-Amoudi et al. (2009) disclose a method for preparation of a dry algal material. Algae (100 g) were extracted with methanol in a Soxhlet apparatus for 8 h. The extract was concentrated under reduced pressure at 60° C., filtered, washed with distilled water, and stored in the dark at 4° C. Fractionation of extracts by centrifugation yielded two fractions (F1 and F2). Fractions were extracted with MeOH—CHCl3. The residue was then sequentially extracted with MeOH—CHCl3 and the final volume was measured and noted as fraction (F2). Samples of each fraction were tested for their hydrolyzed chemical composition.

Barbarino et al. (2005) evaluated eight different methods of protein extraction from plant biomass. 50 mg of freeze-dried algal sample were manually ground with a mortar and pestle. Two different volumes of water were tested (1.0 and 4.0 mL) as well as two different incubation periods of samples in the water (6 h and 12 h). In all the cases samples were kept at 4° C. during the incubation period. After the incubation period, suspensions were centrifuged at 4° C., 15000 g for 20 minutes. Supernatants were collected for protein assay and the pellets re-extracted with 1.0 mL 0.1 N NaOH with 0.5%-mercaptoethanol (v/v). The mixture of NaOH and pellets was kept at room temperature for 1 h with occasional manual shaking and then centrifuged at 21° C., 15,000 g for 20 minutes. The second supernatants were combined with the first ones and the pellets were discarded. The final volume of the extract was between 2.0 and 9.0 mL.

Ursu et al. (2014) discloses a method for biomass extraction from frozen Chlorella vulgaris (28% dry matter). The biomass was thawed and then diluted to obtain a suspension containing 1.3% w/w biomass (dry weight). In order to limit protein damage during extraction, the temperature was maintained at 20° C. A high pressure cell disrupter was employed to release the intracellular proteins. Cell lysis was conducted either at pH=7 or at pH=12 using 1M NaOH before or after the mechanical treatment. After the chemical and/or mechanical treatments, the microalgae suspensions were centrifuged at 5° C., 10,000 g for 30 minutes. Proteins were extracted from the protein-rich supernatant by one of two methods, precipitation at pI or concentration using tangential ultrafiltration. In the first method, after protein solubilisation, the pH of the supernatant was decreased from 12 or 7 to 4 progressively by addition of 1M HCl in order to obtain the pI value of the majority of the proteins. The paste obtained after precipitation and centrifugation was freeze-dried and then stored at room temperature. In the second method, tangential ultrafiltration was performed using a pilot-scale tangential-flow filtration unit. A membrane in PES with a molecular weight cut-off of 300 kDa and 0.1 m² surface of filtration was used for the separation. Ultrafiltration was carried out at room temperature under a fixed transmembrane pressure of 1.5 bar. Approximately 5 L of raw material (supernatant from extractions at pH 7 or pH 12) were concentrated five times by tangential ultrafiltration.

The methods known in the art for fractionating green plant biomass to obtain a water-soluble protein-rich concentrate (RPPRM-WS) suffer from a number of disadvantages. In these methods, the biomass is treated with chemicals that are used as cell wall lysis agents, which remain as an impurity in the resulting RPPRM-WS. In many cases, the level of impurities remaining in the RPPRM-WS is very difficult to control, and the impurities can have the effect of making the RPPRM-WS unusable for further processing or for use as a starting material for synthesis of new products. The use in these methods of processing auxiliaries, and the requirement that the RPPRM-WS undergo additional purification before it can be used as a raw material have the effect of making the RPPRM-WS more costly than it would be if a less complicated process were available for its production.

Thus, a process for producing a protein concentrate from green plant biomass, particularly de-chlorophyllized green plant biomass, that avoids these disadvantages, in particular the requirement of treatment with reagents other than inexpensive and environmentally friendly solvents, remains a long-felt, yet unmet, need.

### SUMMARY OF THE INVENTION

The present invention is designed to meet this long-felt need. The present invention discloses a method for production of a protein concentrate from dechlorophyllized green biomass, particularly green biomass derived from aquatic plants, in which the water-soluble protein fraction is extracted efficiently and without the need of any reagents other than the solvents with which the plant matter comes into contact.

It is an object of the present invention to disclose a method for producing a water-soluble protein concentrate from plant biomass, comprising: obtaining dry de-chlorophyllized plant matter from said plant biomass; treating said dry de-chlorophyllized plant matter with water, thereby at least partially dissolving water-soluble protein content of said dry de-chlorophyllized plant matter and preparing an aqueous suspension of said dry de-chlorophyllized plant matter; separating said aqueous suspension of said dry de-chlorophyllized plant matter into a first neutral extract and a wet solid; and, drying said first neutral extract, thereby yielding a water-soluble protein concentrate.

It is an object of the present invention to disclose a method for producing a water-soluble protein concentrate from plant biomass, comprising: obtaining from said plant biomass dry de-chlorophyllized plant matter comprising water-soluble protein content; treating said dry de-chlorophyllized plant matter with water, thereby yielding (a) an aqueous solution comprising at least part of said water-soluble protein content of said dry de-chlorophyllized plant matter; and (b) a suspension of de-chlorophyllized plant matter in said aqueous solution; separating said suspension into a first neutral extract and a wet solid; and, drying said first neutral extract, thereby yielding a water-soluble protein concentrate.

It is a further object of this invention to disclose such a method as defined in any of the above, comprising washing said wet solid with water, thereby producing a second neutral extract; concentrating said second neutral extract; and, drying said second neutral extract, thereby yielding additional water-soluble protein concentrate, i.e. a second crop of said water-soluble protein concentrate. In some embodiments of the method, it comprises washing said wet solid with water, thereby producing a second neutral extract; combining said first neutral extract and said second neutral extract prior to said step of drying said first neutral extract, thereby producing a combined neutral extract; wherein said step of drying said first neutral extract comprises drying said combined neutral extract, thereby yielding a water-soluble protein concentrate. In some embodiments of the method, said step of washing said wet solid with water comprises washing said wet solid with water at a solid:water ratio of 1:3 by volume. In some preferred embodiments of the method, said step of washing said wet solid with water comprises washing with successive aliquots of water until an aliquot is produced that is characterized by a concentration of dissolved material of less than 0.1% by weight. In some preferred embodiments of the method, said step of concentrating said second neutral extract comprises concentrating said second neutral extract until said second neutral extract is characterized by a dissolved solid content of not less than 5%. In some preferred embodiments of the method, said step of concentrating said second neutral extract comprises concentrating said second neutral extract until said second neutral extract is characterized by a dissolved solid content of not less than 3%. In some preferred embodiments of the method, said step of concentrating said second neutral extract comprises concentrating said second neutral extract until said second neutral extract is characterized by a dissolved solid content of not less than 10%.

It is a further object of this invention to disclose the method as defined in any of the above, wherein said plant biomass is selected from the group consisting of leaves and fronds.

It is a further object of this invention to disclose the method as defined in any of the above, wherein said plant biomass is obtained from aquatic plants. In some embodiments of the method, said plant biomass is obtained from aquatic plants selected from the group consisting of algae, microalgae, and duckweed. In some preferred embodiments of the method, said plant biomass is obtained from duckweed. In some particularly preferred embodiments of the method, said duckweed is selected from the group consisting of Lemna gibba, Spirodela polyrrhiza, Spirodela punctuata, Wolffia arrhiza, Wolffia columbiana, and Wolffia globosa.

It is a further object of this invention to disclose the method as defined in any of the above, wherein said method does not comprise any step in which said plant biomass contacts a solvent that is not approved for use in food or food production.

It is a further object of this invention to disclose the method as defined in any of the above, wherein said method does not comprise any step in which a chemical lysis agent is used.

It is a further object of this invention to disclose the method as defined in any of the above, wherein said step of obtaining dry de-chlorophyllized plant matter comprises: drying said plant biomass, thereby producing dried plant biomass; grinding said dried plant biomass, thereby producing ground dried plant biomass; extracting chlorophyll from said ground dried plant biomass, thereby producing de-chlorophyllized plant matter; and, drying said de-chlorophyllized plant matter, thereby obtaining dry de-chlorophyllized plant matter.

It is a further object of this invention to disclose the method as defined in any of the above, wherein said step of obtaining dry de-chlorophyllized plant matter comprises drying said plant biomass in the absence of light at a temperature not exceeding 50° C. In some preferred embodiments of the method, said step of obtaining dry de-chlorophyllized plant matter comprises drying said plant biomass in the absence of light at a temperature not exceeding 45° C. In some more preferred embodiments of the invention, said step of obtaining dry de-chlorophyllized plant matter comprises drying said plant biomass in the absence of light at a temperature not exceeding 40° C.

It is a further object of this invention to disclose the method as defined in any of the above, wherein said step of grinding said dried plant biomass comprises grinding said dried plant biomass in a ball mill.

It is a further object of this invention to disclose the method as defined in any of the above, wherein said step of grinding said dried plant biomass comprises grinding said dried plant biomass at a temperature not exceeding 30° C.

It is a further object of this invention to disclose the method as defined in any of the above, wherein said step of grinding said dried plant biomass comprises grinding said dried plant biomass to a powder characterized by a maximum particle diameter of 200 μm. In some preferred embodiments of the method, said step of grinding said dried plant biomass comprises grinding said dried plant biomass to a powder characterized by a maximum particle diameter of 150 μm. In some preferred embodiments of the method, said step of grinding said dried plant biomass comprises grinding said dried plant biomass to a powder characterized by a maximum particle diameter of 100 μm.

It is a further object of this invention to disclose the method as defined in any of the above, wherein said step of separating said aqueous suspension of said dry de-chlorophyllized plant matter into a first neutral extract and a wet solid comprises centrifuging said aqueous suspension.

It is a further object of this invention to disclose the method as defined in any of the above, wherein said water is demineralized water characterized by a conductance of less than 4 μS.

It is a further object of this invention to disclose the method as defined in any of the above, wherein said step of treating said dry de-chlorophyllized plant matter with water comprises treating for 2-12 hours. In some preferred embodiments of the invention, said step of treating said dry de-chlorophyllized plant matter with water comprises treating for 3-8 hours. In some particularly preferred embodiments of the invention, said step of treating said dry de-chlorophyllized plant matter with water comprises treating for 4-6 hours.

It is a further object of this invention to disclose the method as defined in any of the above, wherein said step of treating said dry de-chlorophyllized plant matter with water comprises treating dry de-chlorophyllized plant matter with water at a temperature in the range of 20-80° C. In some preferred embodiments of the invention, said step of treating said dry de-chlorophyllized plant matter with water comprises treating dry de-chlorophyllized plant matter with water at a temperature of in the range of 30-70° C. In some particularly preferred embodiments of the invention, said step of treating said dry de-chlorophyllized plant matter with water comprises treating dry de-chlorophyllized plant matter with water at a temperature of in the range of 40-60° C.

It is a further object of this invention to disclose the method as defined in any of the above, wherein said first neutral extract produced in said step of separating said aqueous suspension of said dry de-chlorophyllized plant matter into a first neutral extract and a wet solid comprises a solid content in the range of 5-10% by weight.

It is a further object of this invention to disclose the method as defined in any of the above, wherein said first neutral extract produced in said step of separating said aqueous suspension of said dry de-chlorophyllized plant matter into a first neutral extract and a wet solid comprises a solid content in the range of 1-3% by weight.

It is a further object of this invention to disclose the method as defined in any of the above, wherein said first neutral extract produced in said step of separating said aqueous suspension of said dry de-chlorophyllized plant matter into a first neutral extract and a wet solid comprises a solid content in the range of 0.5-2.5% by weight.

It is a further object of this invention to disclose the method as defined in any of the above, wherein at least one of said steps of drying said neutral extract comprises drying by a method selected from the group consisting of spray drying and freeze drying.

It is a further object of this invention to disclose the method as defined in any of the above, comprising drying said wet solid after all steps of producing neutral extracts have been completed, thereby producing dry fibrous material. In some preferred embodiments of the invention, said step of drying said wet solid comprises drying said wet solid in a hot air dryer at a temperature of between 75° C. and 85° C. In some preferred embodiments of the invention, said step of drying said wet solid comprises drying said wet solid until said wet solid is characterized by a moisture content of less than 15%. In some embodiments of the invention, said step of drying said wet solid is followed by a step of grinding said dry fibrous material. In some preferred embodiments of the invention, said step of grinding comprises grinding until said dry fibrous material is characterized by a maximum particle diameter of less than 1 mm.

It is a further object of this invention to disclose the method as defined in any of the above, wherein said plant biomass does not comprise seeds.

It is a further object of this invention to disclose a water-soluble protein concentrate produced from plant biomass, wherein said organic biomass does not comprise seeds.

It is a further object of this invention to disclose such a water-soluble protein concentrate, wherein said water-soluble protein concentrate is partially water-soluble. It is a further object of this invention to disclose such a water-soluble protein concentrate, wherein said water-soluble protein concentrate is entirely water-soluble.

It is a further object of this invention to disclose a water-soluble protein concentrate as defined in any of the above, wherein said water-soluble protein concentrate comprises proteins having an average molecular weight of less than 12,000 Da. In some preferred embodiments of the invention, said water-soluble protein concentrate comprises proteins having an average molecular weight of less than 8,000 Da.

It is a further object of this invention to disclose a water-soluble protein concentrate as defined in any of the above, wherein said water-soluble protein concentrate is characterized, when in a 25° C. aqueous solution at a concentration of 0.5-1.5%, by a conformational transition from random coil to rod.

It is a further object of this invention to disclose a water-soluble protein concentrate as defined in any of the above, wherein said water-soluble protein concentrate is characterized by an organic matter content of greater than 85% by weight on a dry basis. In some preferred embodiments of the invention, said water-soluble protein concentrate is characterized by an organic matter content of greater than 90% by weight on a dry basis. In some especially preferred embodiments of the invention, said water-soluble protein concentrate is characterized by an organic matter content of greater than 95% by weight on a dry basis.

It is a further object of this invention to disclose a water-soluble protein concentrate as defined in any of the above, wherein said water-soluble protein concentrate is characterized by total nitrogen content and by a crude protein content calculated as 6.25 times the total nitrogen content of 30-55% on an organic matter dry basis.

It is a further object of this invention to disclose a water-soluble protein concentrate as defined in any of the above, wherein said crude protein content is 40-70% on an organic matter dry basis.

It is a further object of this invention to disclose a water-soluble protein concentrate as defined in any of the above, wherein said water-soluble protein concentrate is characterized by a moisture content of between 10% and 15% by weight.

It is a further object of this invention to disclose a water-soluble protein concentrate as defined in any of the above, wherein said water-soluble protein concentrate is characterized by a moisture content of between 7% and 12% by weight.

It is a further object of this invention to disclose a water-soluble protein concentrate as defined in any of the above, wherein said water-soluble protein concentrate is characterized by a moisture content of between 3% and 6% by weight.

It is a further object of this invention to disclose a water-soluble protein concentrate as defined in any of the above, wherein said water-soluble protein concentrate is characterized by an ash content of between 10% and 15% by weight.

It is a further object of this invention to disclose a water-soluble protein concentrate as defined in any of the above, wherein said water-soluble protein concentrate is characterized by an ash content of between 8% and 12% by weight.

It is a further object of this invention to disclose a water-soluble protein concentrate as defined in any of the above, wherein said water-soluble protein concentrate is characterized by an ash content of between 3% and 5% by weight.

It is a further object of this invention to disclose a water-soluble protein concentrate as defined in any of the above, wherein said water-soluble protein concentrate is characterized by a carbohydrate content, calculated as organic compounds without nitrogen, of less than 70% on an organic matter dry basis. In some embodiments of the invention, said water-soluble protein concentrate is characterized by a carbohydrate content, calculated as organic compounds without nitrogen, of less than 50% on an organic matter dry basis.

It is a further object of this invention to disclose a water-soluble protein concentrate as defined in any of the above, wherein said water-soluble protein concentrate is soluble over a pH range of 2-12.

It is a further object of this invention to disclose a water-soluble protein concentrate as defined in any of the above, produced according to the method as defined in any of the above.

### BRIEF DESCRIPTION OF THE DRAWINGS

The invention will now be described with reference to the drawings, wherein

FIGS. 1A and 1B present schematic illustrations of methods for derivatizing and activating, respectively, protein concentrates herein disclosed;

FIG. 2 presents a schematic illustration of chemical processing of protein concentrates herein disclosed to form non-food materials;

FIG. 3 presents results of tangential flow filtration of a neutral extract solution prepared by water treatment of dry de-chlorophyllized plant material at 50° C. for 4 hours; and,

FIG. 4 presents the relationship between the solution concentration and the reduced viscosity for a solution of the protein concentrate of the present invention.

### DETAILED DESCRIPTION OF THE PREFERRED EMBODIMENTS

In the following description, various aspects of the invention will be described. For the purposes of explanation, specific details are set forth in order to provide a thorough understanding of the invention. It will be apparent to one skilled in the art that there are other embodiments of the invention that differ in details without affecting the essential nature thereof. Therefore the figures and examples provided in the invention are to be considered exemplary and not limiting, and the invention is to be understood as limited only as indicated in the accompanying claims, with the proper scope determined only by the broadest interpretation of said claims.

Unless otherwise noted, all concentrations disclosed herein are given on a w/w basis.

The concentrated protein plant materials of the instant invention can be produced from any kind of green plant biomass. In preferred embodiments, the plant biomass is harvested from aquatic environments (marine or fresh water). In more preferred embodiments, the biomass is harvested from algae or duckweeds. In yet more preferred embodiments, duckweeds are used as the source of the plant biomass. In even more preferred embodiments, duckweed of genus Wolffia is used, and in the most preferred embodiments, the source of the biomass is Wolffia globosa.

Typical proximate analyses of the chemical composition of some common duckweed species are given in Table 1. All concentrations are given as percentages. The crude protein content was calculated as 6.25× the nitrogen content, and the carbohydrate content as 100 minus the sum of the moisture, fat, fiber, and ash.

TABLE 1

| | | | Fat | | | |
|---|---|---|---|---|---|---|
| | Mois- | Crude | (Ether | Crude | | Carbohy- |
| Species | ture | Protein | Extract) | Fiber | Ash | drate |
| L. gibba | 4.6 | 25.2 | 4.7 | 9.4 | 14.1 | 46.6 |
| S. punctata | 5.2 | 28.7 | 5.5 | 9.2 | 13.7 | 42.9 |
| S. polyrrhiza | 5.1 | 29.1 | 4.5 | 8.8 | 15.2 | 42.4 |
| W. columbiana | 4.8 | 36.5 | 6.6 | 11 | 17.1 | 28.8 |
| W. arrhiza | 5.3 | 20.4 | 4.6 | 11.6 | 17.6 | 45.8 |

The inventive process uses dry de-chlorophyllized plant matter, which can be prepared by any method known in the art.

In preferred embodiments of the invention, the dry de-chlorophyllized plant matter is prepared according to the following protocol. First, raw plant biomass (preferably fresh) is washed to remove any dirt or foreign material and then dried. Any method for drying the biomass known in the art can be used. Preferably, the drying is done in the dark. In order to prevent thermal degradation of plant pigments, the drying is done at a fairly low temperature, preferably below 50° C., more preferably below 45° C., and most preferably below 40° C.

The dried raw plant biomass is then ground. The grinding is preferably performed in a ball mill, and preferably below 30° C. In preferred embodiments of the invention, the dried raw biomass is ground to a powder having a maximum particle diameter of 200 μm. In more preferred embodiments of the invention, the dried raw biomass is ground to a powder having a maximum particle diameter of 150 μm. In the most preferred embodiments of the invention, the dried raw biomass is ground to a powder having a maximum particle diameter of 100 μm.

The chlorophyll can be removed from the ground dried raw biomass by any method known in the art. In preferred embodiments of the invention, it is extracted by Soxhlet extraction under vacuum using a water-miscible organic solvent. In more preferred embodiments of the invention, the organic solvent is one that is not poisonous to humans. In yet more preferred embodiments of the invention, a food-grade solvent is used. In the most preferred embodiments of the invention, the chlorophyll is extracted using ethanol as the solvent.

Following the extraction of the chlorophyll, the de-chlorophyllized plant material is dried to remove the solvent used to extract the chlorophyll.

The dried de-chlorophyllized plant material is treated with water, preferably demineralized water with a conductance of less than 4 μS. Enough water is added to the de-chlorophyllized plant matter to produce an aqueous suspension. In some preferred embodiments of the invention, the suspension comprises a plant matter/water ratio of 5:95 by weight on a dry matter basis. In some more preferred embodiments of the invention, the suspension comprises a plant matter/water ratio of 10:90 by weight on a dry matter basis. In the most preferred embodiments of the invention, the suspension comprises a plant matter/water ratio of 20:80 by weight on a dry matter basis. The plant material is kept in contact with the water for a predetermined time. In some preferred embodiments of the invention, this treatment lasts between 2 and 12 hours. In some more preferred embodiments of the invention, this treatment lasts between 3 and 8 hours. In the most preferred embodiments of the invention, this treatment lasts between 4 and 6 hours. In typical embodiments of the invention, the temperature of the suspension is maintained between 20° C. and 80° C. during the treatment. In more preferred embodiments of the invention, the temperature of the suspension is maintained between 30° C. and 70° C. during the treatment. In the most preferred embodiments of the invention, the temperature of the suspension is maintained between 40° C. and 60° C. during the treatment.

After the de-chlorophyllized plant material has been treated with water, the suspension is separated, preferably by centrifugation, most preferably by 5000 g centrifugation, into a liquid fraction and a wet solid fraction.

The liquid fraction, also known as the neutral extract, contains water soluble protein (RPPRM-WS) extracted from the plant material during the treatment with water. In some embodiments of the invention, the solid fraction of this first neutral extract is in the range 0.5-2.5%. In some embodiments of the invention, the solid fraction of the neutral extract is in the range 1-3%. In some embodiments of the invention, the solid fraction of the neutral extract is in the range 5-10%.

The wet solid fraction remaining after removal of the liquid fraction, also known as the crude neutral fraction, comprises de-chlorophyllized fiber. In some preferred embodiments of the invention, the wet solid is washed with water and the washing water separated from the wet solid fraction (e.g. by 5000 g centrifugation at 10° C.) to produce a second neutral extract. In some embodiments of the invention, the washing is performed three times at a fiber:water ratio of 1:3 by volume. In some preferred embodiments of the invention, the washing is performed with multiple aliquots of water applied in succession until the supernatant washing water has a dissolved solids content of less than 0.1%.

In preferred embodiments of the invention, the neutral extracts are concentrated, preferably by evaporation in vacuo (typically at a pressure of 40 mbar and a temperature of 40° C.). In some embodiments of the invention, the first and second neutral extracts are combined prior to the step of concentrating them. The neutral extracts are concentrated until the dissolved solids reach a predetermined minimum concentration. In some embodiments of the invention, this concentration is 3%. In some preferred embodiments of the invention, it is 5%. In some more preferred embodiments of the invention, it is 10%.

The concentrated neutral extract is then dried. Any method of drying known in the art can be used. In preferred embodiments, spray drying or freeze drying is used. The resulting solid mass is generally in powder form and comprises a concentrate of the water-soluble protein from the de-chlorophyllized plant material.

In some embodiments of the invention, the wet solid fraction, comprising fibrous material from which water-soluble protein has been extracted, is dried following the washing with water. In typical embodiments, it is dried in a hot air oven at a temperature of about 75° C.-85° C. In preferred embodiments, the drying is performed until the moisture content falls below a predetermined level. In particularly preferred embodiments, the wet solid fraction is dried until the moisture content is less than 15%. The dried fibrous material can then be ground, preferably to a granular mass with particles having a maximum diameter of less than 1 mm, and stored for other uses.

The protein concentrate produced by this method is a water-soluble composition comprising a mixture of substances. Typically, the average molecular weight is less than 12,000 Dalton; in preferred embodiments, the average molecular weight is less than 8,000 Dalton.

Moreover, the protein in the concentrate can be seen to have at least partially undergone a conformational transition from random coil to rod when a dilute solution (0.5-1.5% protein concentrate) is prepared at 25° C.

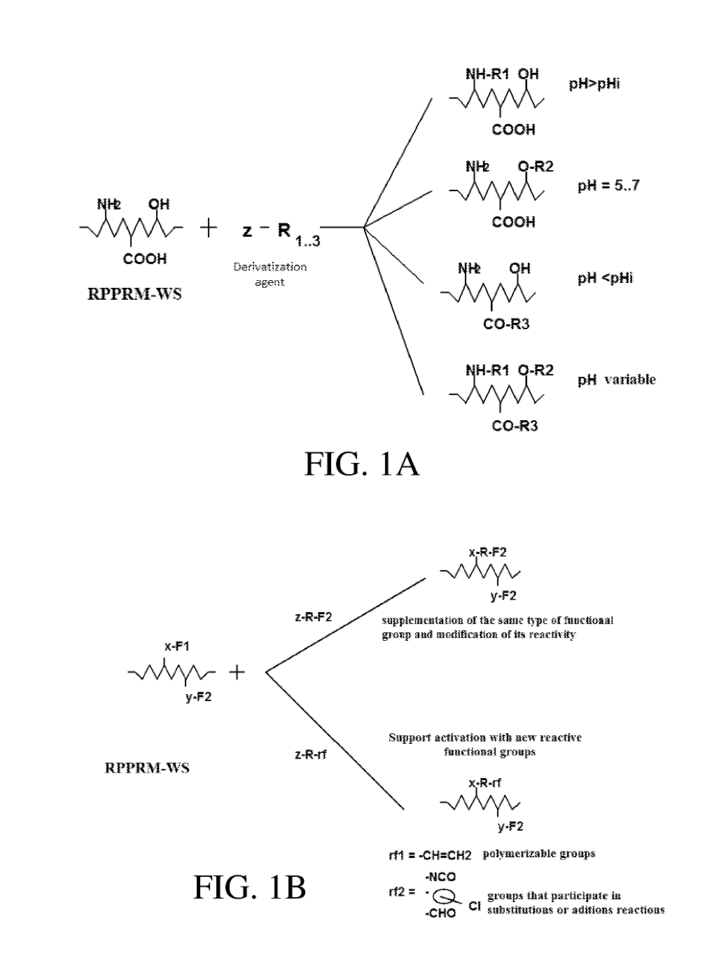

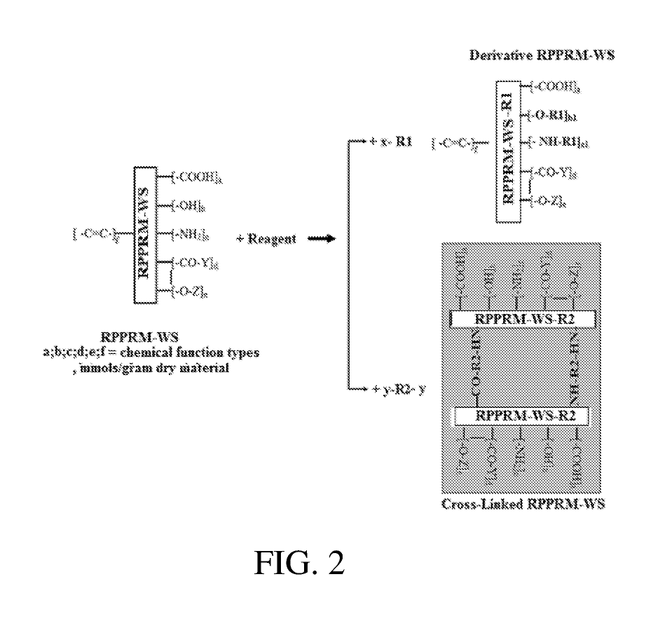

The water-soluble protein concentrate can be used as raw materials for obtaining end products with novel three-dimensional configurations that can be based on covalent or non-covalent bonds. Reference is now made to FIGS. 1A, 1B, and 2, which present schematic illustrations of method of derivatizing, activating, and chemical processing, respectively, of wholly water-soluble protein-rich plant-containing raw materials (RPPRM-WS). The process illustrated in FIG. 2 produces non-food items such as cross-linked hydrogels.

Chemical processing of the protein concentrates herein disclosed may be performed in a variety of environments. Non-limiting examples include aqueous environments and organic solvents at temperatures that are typically between 20° C. and 80° C. Non-limiting examples chemical transformations that can be performed on the protein concentrates of the instant invention include nucleophilic substitution, addition reactions, and free radical polymerization.

The following non-limiting examples are presented in order to assist a person of ordinary skill in the art in understanding how to make and use the invention herein disclosed.

In all of the examples presented, the biomass starting material used was obtained from the duckweed species Wolffia globosa cultivated by Hino-man Ltd. (Israel). The plants were harvested, washed with demineralized water to remove dirt and foreign materials, and dried in a current of warm (40° C.) air using an Ezidri Ultra FD 1000 air dryer obtained from Food Dehydrators (Israel).

The dry green plant material was then de-chlorophyllized by extraction by ethanol according to the procedure disclosed in International (PCT) Pat. Appl. Pub. No. WO2015/145431. The crude de-chlorophyllized plant material obtained after the ethanol extraction was then dried using a Buchi rotary evaporator operated at 200 mbar pressure and 80° C.

A proximate analysis of the chemical composition of the de-chlorophyllized plant material is given in Table 2. All amounts are given in percent by weight.

TABLE 2

| Component | Amount |
|---|---|
| Moisture | 4.53 |
| Ash | 8.11 |
| Crude protein | 72.3 |
| Fats | 0 |
| Carbohydrate | 15.06 |

### Example 1

10 grams of de-chlorophyllized dried Wolffia globosa prepared as described above suspended in 50 ml demineralized water and 450 ml demineralized water (0.4 μS) preheated to 50° C. were added under stirring at a speed of 200 rpm to a 1 liter glass double jacketed reactor (extraction reactor) equipped with an anchor type Teflon stirrer, an overhead stirrer, and a thermometer. A condenser and thermostatic water bath with recirculation were added to the extraction reactor. The resulting suspension was mixed for 4 hours at 50° C., cooled to room temperature, and discharged from the extraction reactor, after which it was separated by vacuum filtration using a Buchner funnel and polyester net with a pore diameter of 100 microns. The separation produced 360 ml of extract solution (neutral extract) and 146 g of insoluble wet solid. The neutral extract solution was found to contain 1.92 g of dissolved solids, as evaluated by a gravimetric method performed on 10 ml aliquots of the solution (average of 3 replicates). The solution was dried at 105° C. for 4 hours using an oven with forced-air convection. The remaining solution of extract was freeze-dried by using a lyophilizer (FreeZone, Labconco). 1.89 g of solid “Rich Plant Protein Raw Material-Water Soluble” (RPPRM-WS-1) was obtained.

A proximate chemical analysis was performed on this material. The ash (inorganic material) content was determined by the ignition method [Santisteban J. I. 2004]. Crude protein (CP) was determined as 6.25%×the nitrogen content. Carbohydrate (CH) content was determined from the formula 100=Ash+M+Cp+Fat+CH. Bradford protein (BP) was determined by UV spectroscopy using the calibration curve obtained from treatment of samples of the solutions prepared with human serum albumin (BSA) and treated with reactive Bradford. The results obtained from the analysis are presented in Table 3. All concentrations are given in percent by weight.

TABLE 3

| Component | Amount |
|---|---|
| Moisture | 3 |
| Ash | 4.3 |
| Fats | 0 |
| Crude protein | 69.2 |
| Bradford protein | 38.6 |
| Carbohydrate | 23.5 |

Sufficient RPPRM-WS-1 was added to water buffered to a predetermined pH to make up a 0.5% solution. The components were centrifuged to 5,000 g at a temperature of 20° C. The RPPRM-WS-1 completely dissolved over the entire pH range 2-12.

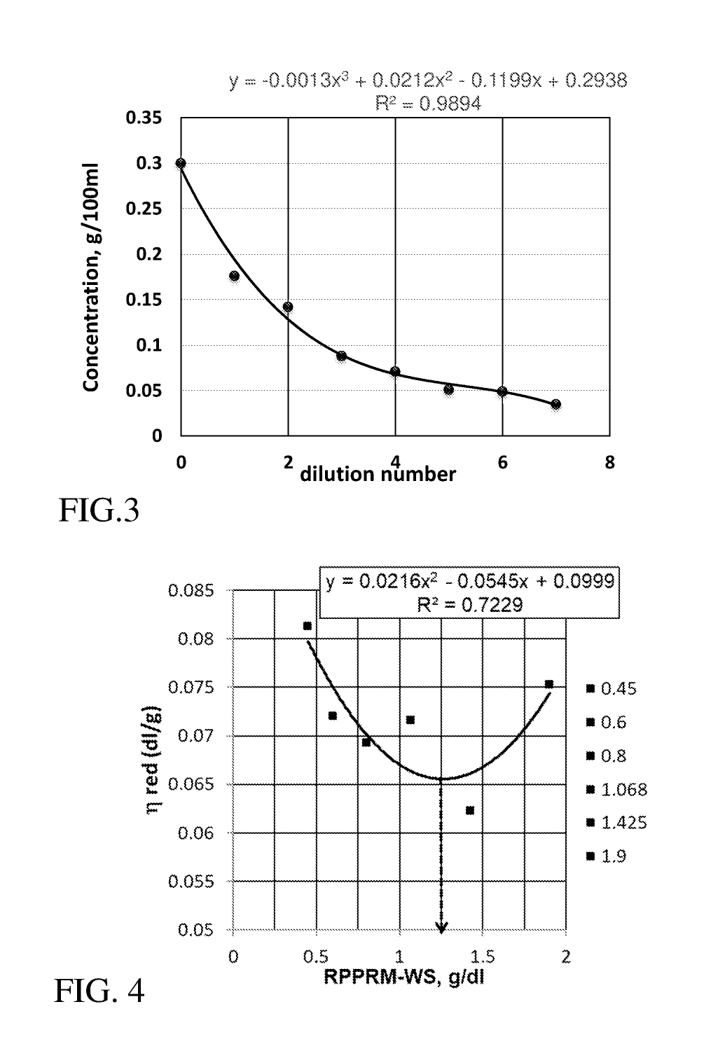

The average molecular mass of the protein concentrate was determined using tangential flow filtration (TFF) by dilution at constant volume using a Minimate TFF apparatus obtained from Pall. For this purpose has been prepared a solution of RPPRM-WS-1 of 0.5% concentration in demineralized water using as medium a filter membrane of 12 kDa. Reference is now made to FIG. 3, which presents the results obtained from the application of TFF. The TFF results indicate the protein concentrate comprises water soluble compounds with average molecular mass lower than 12,000 Da.

A viscosimetric method was used to confirm the coil-rod conformational transition [Tsujita Y. et al 1979., Mark J. E. 2007]. The viscosity of a 1.89% solution of RPPRM-WS-1 in demineralized water (0.4 μS) was determined by using an Ubbelohde viscometer with 1 A capillary (time for demineralized water=90 s) held at 25° C. by using thermostatic viscometer bath VB-1423 (J.P.Selecta, Spain). The results obtained for the variation of reduced viscosity ρ_{red} function on concentration are shown in FIG. 4. These results demonstrated that the coil to rod conformational transition had indeed occurred.

### Example 2

The influence of the temperature and extraction time on the chemical composition of the protein concentrate was investigated. The results are summarized in Table 4.

TABLE 4

| | | | RPPRM-WS | | | | | | | |
|---|---|---|---|---|---|---|---|---|---|---|
| | | | | | | | Protein | | | |
| | | | | | | Organic | by | | | |
| | | | Insoluble | Soluble | Ash | matter | Kjeldahl | Protein | | |
| | Extraction factors | | %; | %; | %; | %; | %; | by | Carbohy- | Non- |
| | T | t | g/100 g | g/100 g | g/100 g | g/100 g | g/100 g | Bradford | drate | protein |
| Sample code | ° C. | hours | de-chl. | de-chl. | extr. | de-chl. | extr | % | % | % |
| RP-WS-2 | 80 | 4 | 75.60 | 24.40 | 4.10 | 23.40 | 68.15 | 19.23 | 7.45 | 48.92 |
| RP-WS-3 | 60 | 2 | 74.58 | 25.42 | 4.00 | 24.40 | 48.31 | 30.87 | 12.61 | 17.46 |
| RP-WS-4 | 40 | 6 | 75.65 | 24.35 | 3.80 | 23.42 | 76.37 | 33.61 | 5.54 | 42.77 |
| RP-WS-5 | 60 | 4 | 76.27 | 23.73 | 3.90 | 22.80 | 69.38 | 35.10 | 6.98 | 34.29 |
| RP-WS-6 | 20 | 12 | 81.52 | 18.48 | 4.20 | 17.70 | 65.16 | 23.62 | 6.17 | 41.54 |

1.-20. (canceled)

21. A method for producing a water-soluble protein concentrate from plant biomass, comprising:
obtaining from said plant biomass dry de-chlorophyllized plant matter comprising water-soluble protein content;
treating said dry de-chlorophyllized plant matter with water, thereby yielding:
an aqueous solution comprising at least part of said water-soluble protein content of said dry de-chlorophyllized plant matter; and,
a suspension of de-chlorophyllized plant matter in said aqueous solution;
separating said suspension into a first neutral extract and a wet solid; and,
drying said first neutral extract, thereby yielding a water-soluble protein concentrate.

22. The method of claim 21, comprising:
washing said wet solid with water, thereby producing a second neutral extract;
concentrating said second neutral extract; and,
drying said second neutral extract, thereby yielding additional water-soluble protein concentrate.

23. The method of claim 21, comprising:
washing said wet solid with water, thereby producing a second neutral extract;
combining said first neutral extract and said second neutral extract prior to said step of drying said first neutral extract, thereby producing a combined neutral extract;
wherein said step of drying said first neutral extract comprises drying said combined neutral extract, thereby yielding a water-soluble protein concentrate.

24. The method of claim 21, wherein said plant biomass does not comprise seeds.

25. The method of claim 21, comprising obtaining plant biomass from aquatic plants selected from the group consisting of algae, microalgae, and duckweed.

26. The method of claim 25, wherein said step of obtaining plant biomass comprises obtaining plant biomass from duckweed.

27. The method of claim 21, wherein said method does not comprise any step in which said plant biomass contacts a solvent that is not approved for use in food production.

28. The method of claim 21, wherein said method does not comprise any step in which a chemical lysis agent is used.

29. The method of claim 21, wherein said step of obtaining dry de-chlorophyllized plant matter comprises:
drying said plant biomass, thereby producing dried plant biomass;
grinding said dried plant biomass, thereby producing ground dried plant biomass;
extracting chlorophyll from said ground dried plant biomass, thereby producing de-chlorophyllized plant matter; and,
drying said de-chlorophyllized plant matter, thereby obtaining dry de-chlorophyllized plant matter.

30. The method of claim 29, wherein at least one of the following is true:
said step of drying said plant biomass comprises drying said plant biomass in the absence of light at a temperature not exceeding 40° C.; and,
said step of grinding said dried plant biomass comprises grinding said dried plant biomass to a powder characterized by a maximum particle diameter of 100 μm.

31. The method of claim 21, wherein said step of treating said dry de-chlorophyllized plant matter with water comprises at least one step selected from the group consisting of:
treating dry de-chlorophyllized plant matter with water in a plant matter/water ratio selected from the group consisting of:
5:95 by weight on a dry matter basis;
10:90 by weight on a dry matter basis; and,
20:80 by weight on a dry matter basis;
treating dry de-chlorophyllized plant matter with water at a temperature of in a range selected from the group consisting of:
20-80° C.;
30-70° C.; and,
40-60° C.; and,
treating dry de-chlorophyllized plant matter with water for a time period selected from the group consisting of:
2-12 hours;
3-8 hours; and,
4-6 hours.

32. The method of claim 22, wherein said step of concentrating said second neutral extract comprises a step selected from the group consisting of:
concentrating said second neutral extract until said neutral extract is characterized by a dissolved solid content of not less than 10%;
concentrating said second neutral extract until said neutral extract is characterized by a dissolved solid content of not less than 5%; and,
concentrating said second neutral extract until said neutral extract is characterized by a dissolved solid content of not less than 3%.

33. The method of claim 22, wherein said step of washing said wet solid with water comprises at least one step selected from the group consisting of:
washing said wet solid with water at a solid:water ratio of 1:3 by volume;
washing at a temperature of 10° C.; and,
washing with successive aliquots of water until an aliquot is produced that is characterized by a concentration of dissolved material of less than 0.1% by weight.

34. The method of claim 23, wherein said step of washing said wet solid with water comprises at least one step selected from the group consisting of:
washing said wet solid with water at a solid:water ratio of 1:3 by volume;
washing at a temperature of 10° C.; and,
washing with successive aliquots of water until an aliquot is produced that is characterized by a concentration of dissolved material of less than 0.1% by weight.

35. The method of claim 21, comprising drying said wet solid after all steps of producing neutral extracts have been completed, thereby producing dry fibrous material.

36. The method of claim 34, wherein said step of drying said wet solid is followed by a step of grinding said dry fibrous material.

37. A water-soluble protein concentrate produced from plant biomass, wherein said plant biomass does not comprise seeds, and said water-soluble protein concentrate is characterized by at least one characteristic selected from the group consisting of:
said water-soluble protein concentrate comprises proteins having an average molecular weight of less than 8,000 Da;
said water-soluble protein concentrate comprises a crude protein content of 40-70% on an organic matter dry basis;
said water-soluble protein concentrate comprises a moisture content of between 3% and 6% by weight;
said water-soluble protein concentrate comprises an ash content of between 3% and 5% by weight; and,
said water-soluble protein concentrate comprises a carbohydrate content, calculated as organic compounds without nitrogen, of less than 50% on an organic matter dry basis.

38. The water-soluble protein concentrate of claim 37, wherein said water-soluble protein concentrate is partially water-soluble.

39. The water-soluble protein concentrate of claim 37, wherein said water-soluble protein concentrate is entirely water-soluble.

40. A water-soluble protein concentrate produced from plant biomass, wherein:
said plant biomass does not comprise seeds;
said water-soluble protein concentrate is characterized by at least one characteristic selected from the group consisting of:
said water-soluble protein concentrate comprises proteins having an average molecular weight of less than 8,000 Da;
said water-soluble protein concentrate comprises a crude protein content of 40-70% on an organic matter dry basis;
said water-soluble protein concentrate comprises a moisture content of between 3% and 6% by weight;
said water-soluble protein concentrate comprises an ash content of between 3% and 5% by weight; and,
said water-soluble protein concentrate comprises a carbohydrate content, calculated as organic compounds without nitrogen, of less than 50% on an organic matter dry basis; and
said water-soluble protein concentrate is produced according to the method of claim 21.
